(19) United States
(12) Patent Application Publication
(10) Pub. No.: US 2019/0112670 A1
(43) Pub. Date: Apr. 18, 2019

(54) Use Of Wiskott-Aldrich Syndrome Protein (WASP) As A Biological Marker And In Vitro Method For Monitoring The Progression Of A Hematological Disease

(71) Applicant: Sociedade Beneficente Israelita Brasileira Hospital Albert Einstein, São Paulo (BR)

(72) Inventor: Welbert de Oliveira Pereira, São Paulo (BR)

(21) Appl. No.: 16/158,065

(22) Filed: Oct. 11, 2018

Related U.S. Application Data
(60) Provisional application No. 62/571,442, filed on Oct. 12, 2017.

(51) Int. Cl. C12Q 1/6886; G01N 33/574

(52) U.S. Cl. CPC C12Q 1/6886; G01N 33/57426; G01N 2333/47; C12Q 2600/118; C12Q 2600/158

(57) ABSTRACT

The present invention is in the field of hematological diseases of carcinogenic origin. More specifically, the present invention relates to the use of Wiscott-Aldrich syndrome protein (WASP) as a biological marker of the progression of a hematological disease, in particular leukemias, and to an in vitro method for monitoring the progression of a hematological disease, such as leukemia.

### CROSS REFERENCE TO RELATED APPLICATION

The present application claims the benefit of U.S. Provisional Application No. 62/571,442, filed on Oct. 12, 2017 and fully incorporated herein by reference.

### SEQUENCE LISTING

This application contains a Sequence Listing which has been submitted electronically in ASCII format and is hereby incorporated by reference in its entirety. The ASCII copy, created on Oct. 11, 2018, is named Sequence_Listing_8692_144101_ST25.txt and is 2.6 KB in size.

### FIELD OF THE INVENTION

The present invention is in the field of hematological diseases of carcinogenic origin. More specifically, the present invention relates to the use of Wiscott-Aldrich syndrome protein (WASP) as a biological marker of the progression of a hematological disease, in particular leukemias, and to an in vitro method for monitoring the progression of a hematological disease, such as leukemia.

### BACKGROUND OF THE INVENTION

Leukemia is a cancer of hematopoietic cells, including red and white blood cells. Depending on the category of cells, leukemias are classified as acute or chronic, myeloid or lymphoid. Chronic Myeloid Leukemia (CML) is a hematological malignancy caused by the chromosome translocation t(9:22) (q34; q11), when the c-ABL gene present on chromosome 9 merges with the BR gene of chromosome 22, thus forming a shortened chromosome 22, called the Philadelphia chromosome, and originating BCR-ABL oncogene. The incidence of CML is 1.5 case per 100,000 individuals in the world per year, and about 90% of patients are diagnosed at the onset of the disease.

The BCR-ABL oncogene encodes for BCR-ABL, a cytoplasmatic protein composed of multiple domains that retain the structural features of Bcr and c-Abl proteins. The amino-terminus portion of c-Abl moiety contains SH3 (Src homology 3), SH2 and tyrosine kinase (SH1) domains. These domains can ensure a self-inhibitory structure in which SH3 and SH2 participate in the regulation of SH1 kinase activity. In the first exon of c-Abl (at amino-terminus) there is a myristol group, which binds to tyrosine kinase domain, holding SH2 and SH3 trapped. However, BCR-ABL does not possess this myristol group, which is lost in the chromosome translocation, resulting in high constitutive kinase activity, a key factor for the oncogenic potential of the BCR-ABL protein.

Particularly, BCR-ABL tyrosine kinase activity induces a strong resistance to both intrinsic and extrinsic pathways of apoptoses. Therefore, it is not surprising that the development of specific inhibitors directed to the tyrosine kinase catalytic site of BCR-ABL, such as imatinib mesylate, revolutionized the treatment of CML patients.

The progression of CML comprehends three stages: chronic, accelerated and blast phases. In the first phase, called the chronic phase (CF), the patients present, for the most part, clinical manifestations such as splenomegaly, fadifa, weight loss and leukocytosis with normal platelet count. During the chronic phase, BCR-ABL-positive clones are expanding in the bone marrow and still have the ability to differentiate into mature cells, with no blasts being observed at the periphery. Therefore, this phase is considered to be less aggressive, and potentially silent, since the patient's clinical condition may be confused with several other diseases, such as Wiskott-Aldrich syndrome, for example.

At this early stage, myeloid cells and some circulating lymphoids carry the BCR-ABL oncogene. Further, it is believed that the chronic phase can be divided into two subphases, the initial one as described above, and the late one when there are mutations in the SH1 domain of the ABL portion, as well as DNA breaks due to cytogenetic abnormalities acquired post-, or without treatment, but still with clinical manifestations of the chronic phase.

The disease progresses to the so-called accelerated phase (AP), in which occurs an increase of circulating leukocytes or plaquetosis, persistent splenomegaly, an increase of basophils in the bone marrow and peripheral blood and increased frequency of blasts in the bone marrow (about 20-30%). It is understood that such cytogenetic abnormalities mark the transition from the chronic phase to accelerated phase, in which the leukemic clones respond less to conventional treatments due, in particular, to mutations in the ATP binding site of the SH1 domain of the BCR-ABL protein. During the accelerated phase, patients may or may not respond to treatment and present remission for months or years or the progression to the blast crisis stage.

Albeit widely known, for prognostic purposes, the monitoring of these cytogenetic abnormalities is not efficient, once it does not provide physicians and patients with accurate and quick information about the progression of CML to the accelerated and blastic phases.

In the last stage of the CML, called blast crisis phase (CB), the leukemic clones lose their differentiation ability, leading to the accumulation of blasts in the bone marrow and blood (corresponding to more than 30% of the circulating cells) and the death of the patient within approximately 6 months.

Rarely, patients with blast crisis achieve remission and, in general, they regress later. In these cases, the bone marrow transplantation is the only intervention for CML proven to be effective. However, it should be noted that, in many cases, bone marrow transplantation does not achieve the expected success, causing the patient to develop a graft rejection reaction with varying degrees of severity.

As the blast crisis phase is the most severe and potentially lethal in up to 6 months, assertive therapeutic decisions prior to it are essential. For patients to have a better chance of survival, it is important that the physician understands their therapeutic needs in early stages of the disease for making crucial clinical decision regarding increasing drug dosages or replacement of the treatment, which may be selected from, but not limited to, tyrosine kinase inhibitors (TKIs), such as imatinib, desatinib, nilotinib, bosutinib, ponatinib and variations thereof, chemotherapy, radiotherapy, immunosuppressants, immunomodulatory agents, bone marrow transplantation, among others.

The TKIs are currently the first line treatment, but their effectiveness declines when the patient evolves to advanced phases of leukemia. Thus, even in patients already under treatment, the monitoring of the disease progression is essential.

The progression of CML from initial stages to advanced phases is followed up by clinical aspects and the analysis of BCR-ABL expression in the blood, but both of them are not clear enough to determine the phase transition or to predict if the disease is evolving. In addition, the BCR-ABL expression levels is evaluated by means of Polymerase Chain Reaction (PCR), which may take up to 15 days for the results to be made available, and during this period of time the CML can progress rapidly and lethally, without the physician having time to adjust the patient treatment and delay the progression of the disease.

It is also worth noting that there are leukemias that do not have said chromosome translocation and cannot be easily monitored by the above techniques. For example, acute myeloid leukemia (AML) is a type of leukemia of rapid lethality, for which there are no known molecular markers that can be used to monitor the progression of the disease.

Though, for cancer, and particularly leukemia, fast decision making can define the treatment success and thus influence patient survival rate. Therefore, follow-up tests for disease progression that readily inform the physician and patient about the stage of the disease may improve disease prognosis due to a faster and better decision of the physician regarding the treatment.

In accordance with the aforementioned, the aim of the present invention is to provide simple and less time-consuming means of monitoring the cancer progression, in particular acute and chronic myeloid leukemia, capable of providing clinical results in few days or less than 24 hours to assist physicians to make assertive decisions regarding patient treatment.

In this sense, the inventors have surprisingly found out that Wiskott-Aldrich syndrome protein (WASP) may be used as a biological marker for monitoring the progression of a hematological disease, in particular, leukemia. It was shown that both gene expression and protein levels of WASP inversely correlates with BCR-ABL levels and with the disease progression in patients.

WASP is a molecule present in normal blood cells that is important for efficient action of immune system cells. Mutations or decreased expression of WASP are related to immunological deficiencies in pediatric patients. So far the researchers have dedicated to the study of WASP in the immune response and infections, but not in cancer, especially in leukemia.

Therefore, it is surprising that the present invention relates such protein to the progression of a hematological disease, such as leukemia, and provides a method for monitoring the progression thereof that is quick, allowing the physician to make a fast and assertive decision regarding the treatment prior to the progression of the disease and at a lower cost compared to the conventional methods used in the state of the art.

### SUMMARY OF THE INVENTION

The present invention discloses the inventive use of WASP as a biological marker of the progression of a hematological disease in a subject, such as leukemia, and an in vitro method for monitoring the progression of such disease comprising the steps of (a) measuring the amount of WASP in a biological sample from a subject, and (b) comparing the amount of WASP from step (a) with a reference.

Unlike the biomarkers of the prior art, the present invention provides a method for monitoring the progression of a hematological disease, such as leukemia, that is easier and less expensive, allowing the physician to make fast decisions regarding the patient treatment prior to the aggravation of the disease, thereby increasing survival of said patient.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1: The expression of WASP is suppressed in CML patients comparing to healthy donors (HD). The relative expression of WASP in PBMC was determined by real-time PCR using GAPDH as housekeeping gene (**P<0.01, comparing with HD). Values are plotted as 2-^{ΔΔCt}. ‘t’ student was used as statistical test.

FIG. 2: WASP detection in blood samples from healthy donors (HD) and CML patients. The downregulation of WASP was already observed in chronic phase (CP: p<0.05 comparing to healthy donors), and more evident in advanced phases of the disease (AP and CB: p<0.0001). Patients that responded (responsive) to the treatment presented higher levels of WASP. The patients resistant to the treatment present lower levels comparing to healthy donor and to those in chronic phase (p<0.001) and similar levels of advanced phases. Analysis of variance (ANOVA) and Bonferronni post-test were used to statistical analysis.

FIG. 3: Real-time qPCR were performed to evaluate the expression of WASP, which negatively correlates with BCR-ABL1 expression in CML patients (P<0.0001). Spearman test was used with linear regression for statistic analysis.

FIG. 4: Western blot for WASP protein levels detection. The superior part of the figure shows the detection of BCR-ABL protein in order to distinguish samples derived from CML (K562, BV173, LAMA84 and KCL22) from cell from other leukemia/lymphoma patients (HL60 [acute promyelocitic leukemia], Jurkat [T cell acute lymphoblastic leukemia], SKW6.4 [B-cell leukemia], THP-1 [acute myeloid leukemia], U937 [histiocytic lymphoma]). The inferior part of the figure shows the detection of actin protein as housekeeping (quality control of the technique).

FIG. 5: Stable expression of BCR-ABL1 was induced in HL-60 and Jurkat cell lines by retroviral infection, resulting in downregulation of WASP both at the mRNA and protein levels. GAPDH was used as housekeeping control for qPCR. Western blots show the increased numbers of tyrosine phosphorylated proteins in BCR-ABL-positive HL-60 and Jurkat cell lines, and the complete WASP silencing after BCR-ABL1 expression. Actin protein was used as loading control.

FIG. 6: The relative WASP expression values obtained from patients in AP and BP were divided in two groups according to the median: WASP very low for patients exhibiting a strong WASP downregulation (lower than median), and WASP low for patients presenting a mild WASP suppression (higher than median).

FIG. 7: Patients with higher levels of WASP (grouped in WASP low) presented longer OS (median=61.75 months) comparing with the patients in WASP very low group. Kaplan-Meier was used as statistical test.

FIG. 8: Meta analysis of WAS gene expression in blood samples from healthy donors (control) and AML patients. Dramatic downregulation is observed in AML patient samples comparing to normal control individuals. Red box: high expression of the gene. Green box: lower expression of the gene. Each colum is related to one patient sample. Each line is related to one gene analyzed. The results show that AML cells express less or no WASP (WAS).

### DEFINITIONS

“WASP” or “Wiskott-Aldrich syndrome protein” is an adaptor protein exclusively expressed in hematopoietic cells capable of promoting actin polymerization and which may act as an endogenous inhibitor of the tyrosine quinases (TKs) of LCK, FYN and c-ABL domains.

The term “tyrosine kinase” means a protein capable of phosphorylating the amino acid tyrosine on another protein, which leads to conformational changes and typically activation of that protein.

The terms “LMC” and “chronic myeloid leukemia” mean a hematological neoplasm caused by the presence of BCR-ABL oncogene. This gene arises from the t(9:22) (q34; q11) chromosomal translocation, when the c-ABL gene present on chromosome 9 merges with the BCR gene on chromosome 22, thus forming a shortened chromosome 22 called “Philadelphia chromosome”.

The terms “AML” and “acute myeloid leukemia” mean a type of cancer that affects the bone marrow and blood, leading to a rapid growth of abnormal immature white blood cells called blast cells and decrease in the number of red blood cells and platelets.

The term “chromosomal translocation” means a chromosomal anomaly caused by the rearrangement of parts of non-homologous chromosomes.

The terms “biological marker” or “biomarker” mean a parameter which is objectively measured and evaluated as an indicator of normal biological processes, pathogenetic processes, or pharmacological responses to a therapeutic intervention. A biological marker may be a substance that indicates a particular pathological state or a specific physiological state. The biological marker according to the invention is preferentially gene products such as the transcripts of said gene and the peptides derived from the transcripts of said gene.

The term “progression” means the worsening of a disease or the progressing to late stages of a disease.

The term “survival” means the extension of life beyond a certain limit.

According to the invention, the term “biological sample” means any sample that can be taken from a subject. Alternatively, the biological sample is a sample of blood, bone marrow and cerebrospinal fluid (cerebrospinal fluid).

### DETAILED DESCRIPTION OF THE INVENTION

The present invention is directed to the use of Wiskott-Aldrich syndrome protein (WASP) as a biological marker of the progression of a hematological disease.

According to preferred embodiments of the invention, the hematological disease is leukemia, and more particularly chronic myeloid leukemia (CML) and acute myeloid leukemia (AML).

Another object of the present invention relates to a particular in vitro method for monitoring the progression of a hematological disease in a subject comprising the following steps:

(a) measuring the amount of WASP in a biological sample from said subject; and

(b) comparing the amount of WASP from step (a) with a reference.

Preferably, the hematological disease is leukemia, and more particularly chronic myeloid leukemia (CML) and acute myeloid leukemia (AML).

According to the present invention, the reference is a measurement of the amount of WASP in a sample previously obtained from the subject who suffers from the hematological disease.

According to the present invention, a reduction of the amount of WASP compared to the reference indicates the progression of the hematological disease.

In a preferred embodiment, the biological sample is selected from a blood sample, a bone marrow sample and a cerebrospinal fluid (cerebrospinal fluid) sample. Preferably, the biological sample is a blood sample.

In another preferred embodiment, the amount of WASP is determined by measuring the amount of WASP nucleic acid. Preferably, the amount of WASP is measured by a technique selected from Northern blot, Southern blot, PCR, RT-PCR, qRT-PCR, SAGE and derivatives thereof, nucleic acid arrays, notably cDNA arrays, oligonucleotide arrays and mRNA arrays, and RNA-Seq.

Still in a preferred embodiment, the amount of WASP is determined by qRT-PCR.

According to the present invention, the amount of WASP is determined by measuring the amount of polypeptide. Preferably, the amount of WASP is measured by a technique selected from immunofluorescence, Western blot, dot blot, ELISA or ELISPOT, ECLIA, immunoprecipitation, FRET or BRET techniques, microscopy, flow cytometry, analysis by polyacrylamide gel electrophoresis (SDS-PAGE), HPLC-mass spectrophotometry and liquid chromatography-mass spectrophotometry/mass spectrometry (LC-MS/MS).

In the most preferred embodiment, the amount of WASP is determined by flow cytometry.

The following examples are intended to illustrate the invention with reference to some preferred embodiments, without being limited to the details shown. Rather, various modifications may be done without departing from the scope of the invention.

### EXAMPLES

### Example 1

### Downregulation of WASP in CML Patients and BCR-ABL-Positive Cell Lines

Initially, the expression of WASP in CML patients and in BCR-ABL-positive cell lines was investigated. Thirty-two healthy individual and 85 CML patients (62 CP, 11 AP and 12 crisis blastic phase) were analyzed, from which 39 were responsive to TKIs therapy (imatinib or dasatinib) and 46 were resistant. Most of patients from responsive groups presented a major molecular response after dasatinib therapy.

CML diagnosis of the patients enrolled in this study was confirmed by the demonstration of Philadelphia chromosome in conventional cytogenetics and/or BCR-ABL detection by RT-PCR. Hematologic, cytogenetic and molecular responses were redefined according to the European LeukemiaNet 2013 recommendations.

Peripheral blood mononuclear cells from patients and controls were isolated according to standard protocol with the Ficoll-Hypaque 1077 density technique.

All cell lines K562, LAMA-84, KCL22, BV173, Jurkat, HL-60, SKW6.4, THP-1 and U937 were cultured in RPMI medium 1640 supplemented with 10% fetal bovine serum, 25 mM Hepes, 2 mM L-glutamine, 100 U/ml penicillin and 100 μg/ml streptomycin. HL-60 and K562 cells were obtained from ATCC (Manassas, Va., USA). HL-60.BCR-ABL cells were derived from wild-type HL-60 by retroviral transfection with pSRαMSVp185^{BCR-ABL1}tkneo. K562 were infected with lentivirus vectors to induce expression of WASP.GFP and WIP.mchery, and sorted using FACS ARIA (Becton-Dickinson, Franklin Lakes, N.J., USA) to obtain high purity cell lines.

For western blot, anti-WASP (Santa Cruz Biotechnologies, Dallas, Tex., USA), anti-WIP (Santa Cruz Biotechnologies), anti-c-ABL OP-20 (Oncogene Research Products, La Jolla, Calif., USA), anti-Actin (Sigma Aldrich, St. Louis, Mo., USA) and TRAIL-R2/Fc protein were used.

For qRT-PCR, total RNA was extracted using Trizol (Invitrogen, Carlsbad, Calif., USA). RNA concentration and purity were determined spectrophotometrically by measuring fluorescence at 260 and 280 nm. Three micrograms of RNA was reverse transcribed into cDNA using Superscript III (Invitrogen) transcription reagents according to the manufacturer's instructions. After obtaining the cDNA, gene expression was quantified by qPCR using Platinum SYBRGreen Kit (Invitrogen) in Mx3005P detector equipment (Stratagene, Santa Clara, Calif., USA). The following primers were used: BCR-ABL 5′-TGGGTCCAGCGAGAAGGTT-3′ (forward) (SEQ ID NO. 1) and 5′-GCATTCCGCTGACCATCAAT-3′ (reverse) (SEQ ID NO. 2); GAPDH 5′-GGAGAA GGCTGGGGCTCAT-3′ (forward) (SEQ ID NO. 3) and 5′-TCCTTCCACGATACCAAAGTT-3′ (reverse) (SEQ ID NO. 4); TRAIL 5′-AAGGCTCTGGGCCGCAAAATAAAC-3′ (forward) (SEQ ID NO. 5) and 5′-CCAACTAAAAAGGCCCCGAAAAA-3′ (reverse) (SEQ ID NO. 6); DR4 5′-GTACGCCCTGGAGTGACATC-3′ (forward) (SEQ ID NO. 7) and 5′-CCTCGTAGGAGACCCAAGC-3′ (reverse) (SEQ ID NO. 8); DR5 5′-CTAGCTCCCCAGCAGAGAGT-3′ (forward) (SEQ ID NO. 9) and 5′-GTGGTGCAGGGACTTAGCTC-3′ (reverse) (SEQ ID NO. 10); WASP 5′-GGCTGGTCGGCTGCTCTGGGAACA-3′ (forward) (SEQ ID NO. 11) and 5′-GGTGGTGGGGGTAGCTGGCGTCTGT-3′ (reverse) (SEQ ID NO. 12). Results were given as relative expression represented as 2^{-ΔΔCt}.

For Western blot, protein samples were resolved under reducing conditions. Separated proteins were transferred onto polyvinylidene difluoride membranes and reactions were detected with a suitable secondary antibody conjugated to horseradish peroxidase (Jackson Laboratory, Bar Harbor, Me., USA and Amersham, Arlington, Ill., USA) using enhanced chemiluminescence (Pierce, Rockford, Ill., USA).

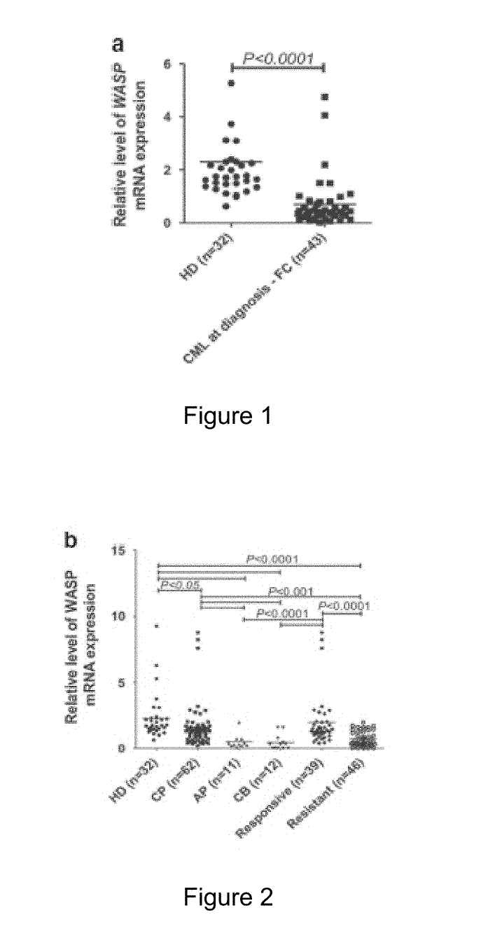

It was found that PBMC from CML patients expressed significantly lower levels of WASP compared to healthy donors (FIG. 1). CML patients in the CP presented lower levels of WASP and its expression was decreased during the progression of the disease to accelerated and blast phases (FIG. 2). Importantly, PBMC from patients resistant to TKI exhibited significantly lower levels of WASP compared to patients responsive to TKI (patients who achieved the complete cytogenetic remission (CCyR) and major molecular remission (MMR) after treatment with imatinib and dasatinib). Patients responsive to TKI were not different from either healthy individuals or CML patients at diagnosis (FIG. 2). These data indicate that WASP expression is linked to CML patient's response to TKIs therapy and to BCR-ABL levels.

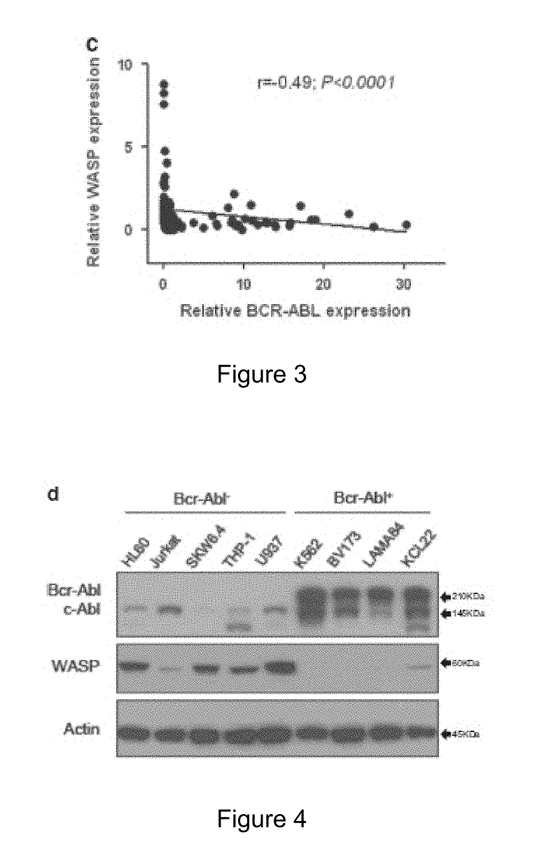

Moreover, the expression levels of WASP and BCR-ABL were inversely correlated (FIG. 3), suggesting that BCR-ABL could potentially be responsible for WASP downregulation. In order to investigate this possible cause-effect relationship, the WASP protein expression in cell lines derived from BCR-ABL-negative leukemia (HL-60, Jurkat, SKW6.4, THP-1 and U937) and BCR-ABL-positive CML patients (K562, BV173, LAMA-84 and KCL22). Unlike BCR-ABL1-negative cells, WASP was strongly downregulated in BCR-ABL-positive cell lines (FIG. 4) further supporting that BCR-ABL is a negative regulator of WASP.

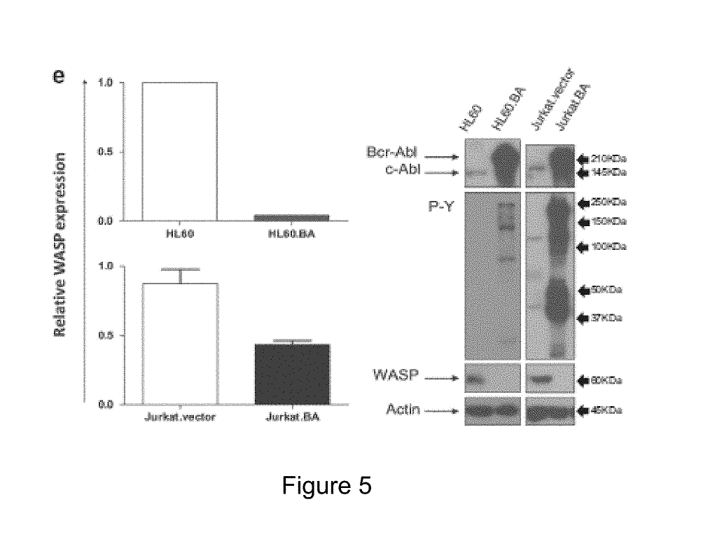

This hypothesis was confirmed by transducing BCR-ABL in the HL-60 and Jukart cell lines, thereby producing HL-60.BCR-ABL and Jukart.BCR-ABL cells. Enforced expression of BCR-ABL induced a strong suppression of WASP, observed at both mRNA and protein levels (FIG. 5). The efficacy of BCR-ABL transduction was confirmed by immunoblot using primary antibodies against c-ABL-BCR-ABL (to verify its expression) or to phosphotyrosine (to verify its activity) (FIG. 5). These results show that expression of BCR-ABL inhibits WASP at both mRNA and protein levels.

### Example 2

### Expression of WASP in PBMC from CML Patients in Advanced Phases and Overall Survival

Next, the biological relevance of decreased levels of WASP for CML patients was investigated. The probability of OS of 31 CML newly diagnosed patients according to WASP expression levels was calculated from CML diagnosis until death or last follow-up using the Kaplan-Meier method and the log-rank test, using IBM SPSS software, version 21 (IBM, Armonk, N.Y., USA). It was also calculated the OS of 23 CML patients in advanced phases (AP and BP) resistant to IM 400 mg daily according to WASP expression levels.

WASP gene expression analysis among healthy individuals and patients' different groups were performed by using the GraphPad Prism software version 7 (GraphPad Software Inc., La Jolla, Calif., USA).

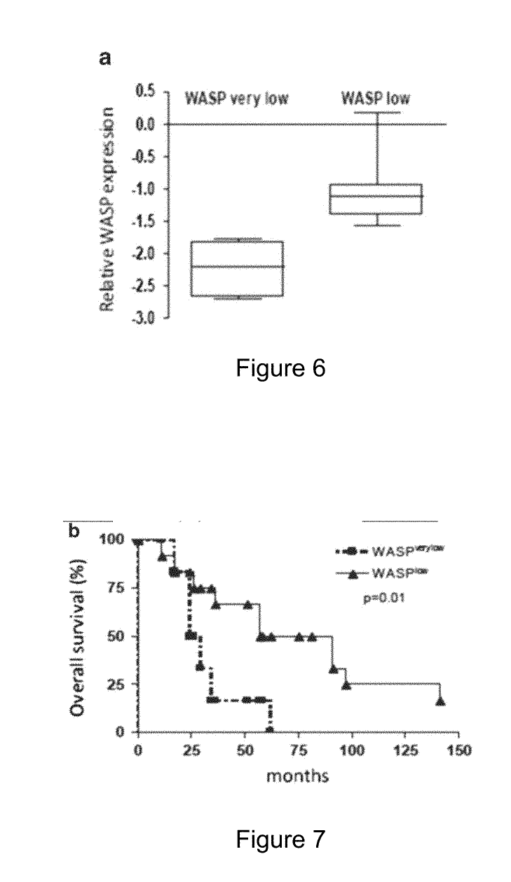

The relationship between the levels of WASP with the overall survival (OS) of the 31 newly diagnosed CML patients and in 23 CML patients at accelerated phase (AP) and blast crisis (BC) was analyzed. For this analysis, the patients were divided into two groups according to the median of WASP expression (FIG. 6). Patients with WASP levels below the median were called WASP BM, and patients with WASP levels above the median were called WASP AM. There was no significant difference in OS according to WASP expression at CML diagnosis (data not shown).

Interestingly, when the OS was calculated just for CML patients in advanced phases, resistant to TKIs and with significantly lower levels of WASP compared to healthy donors, it was observed that milder suppression of WASP (levels above median—WASP low) correlated with longer OS, whereas strong suppression of this gene (levels below median—WASP very low) correlated with poorer OS (FIG. 7). Altogether, these data suggest that the downregulation of WASP by BCR-ABL may be relevant to the CML prognosis, particularly for patients in advanced phases of disease.

### Example 3

### Downregulation of WASP in AML Patients

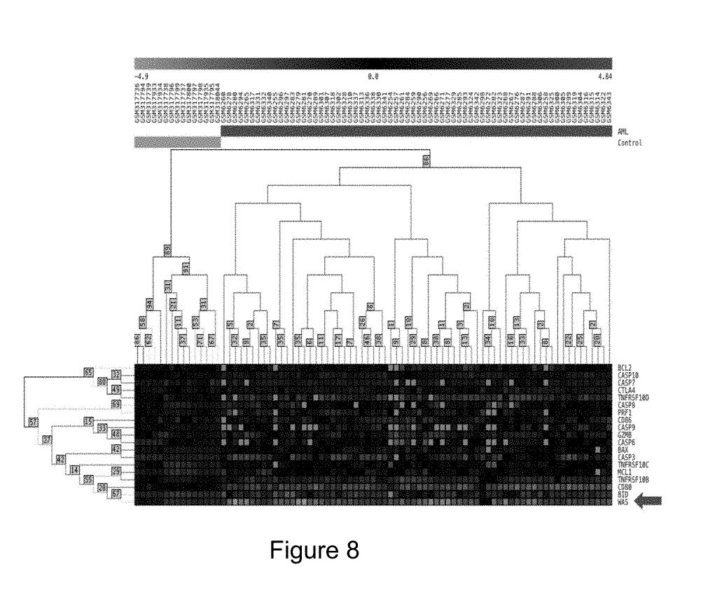

Finally, a meta-analysis using blood samples from healthy donors (control) and AML patients was performed to evaluate whether the WASP expression was also decreased for such type of leukemia. Hence, it was found that AML patients expressed lower levels of WASP compared to healthy donors (FIG. 8).

1. An in vitro method for monitoring the progression of a hematological disease in a subject comprising the following steps:
(a) measuring the amount of WASP in a biological sample from said subject; and
(b) comparing the amount of WASP from step (a) with a reference.

2. The method of claim 1, wherein the hematological disease is leukemia, preferentially, chronic myeloid leukemia (CML) and acute myeloid leukemia (AML).

3. The method of claim 1, wherein the reference is a measurement of the amount of WASP in a sample previously obtained from said subject who suffers from the hematological disease.

4. The method of claim 1, wherein an amount of WASP from step (a) lower than the reference indicates the progression of the hematological disease.

5. The method of claim 1, wherein the biological sample is selected from a blood sample, a bone marrow sample and a cerebrospinal fluid (cerebrospinal fluid) sample.

6. The method of claim 5, wherein the biological sample is a blood sample.

7. The method of claim 1, wherein the amount of WASP is determined by measuring the amount of WASP nucleic acid.

8. The method of claim 7, wherein the amount of WASP is measured by a technique selected from Northern blot, Southern blot, PCR, RT-PCR, qRT-PCR, SAGE and derivatives thereof, nucleic acid arrays, notably cDNA arrays, oligonucleotide arrays and mRNA arrays, and RNA-Seq.

9. The method of claim 8, wherein the amount of WASP is determined by qRT-PCR.

10. The method of claim 1, wherein the amount of WASP is determined by measuring the amount of polypeptide.

11. The method of claim 10, wherein the amount of WASP is measured by a technique selected from immunofluorescence, Western blot, dot blot, ELISA or ELISPOT, ECLIA, immunoprecipitation, FRET or BRET techniques, microscopy, flow cytometry, analysis by polyacrylamide gel electrophoresis (SDS-PAGE), HPLC-mass spectrophotometry and liquid chromatography-mass spectrophotometry/mass spectrometry (LC-MS/MS).

12. The method of claim 11, wherein the amount of WASP is determined by flow cytometry.


SEQUENCE LISTING (12 sequences)

SEQ ID NO: 1 - DNA, 19 bp, Artificial Sequence
  Synthetic construct
tgggtccagcgagaaggtt

SEQ ID NO: 2 - DNA, 20 bp, Artificial Sequence
  Synthetic construct
gcattccgctgaccatcaat

SEQ ID NO: 3 - DNA, 19 bp, Artificial Sequence
  Synthetic construct
ggagaaggctggggctcat

SEQ ID NO: 4 - DNA, 21 bp, Artificial Sequence
  Synthetic construct
tccttccacgataccaaagtt

SEQ ID NO: 5 - DNA, 24 bp, Artificial Sequence
  Synthetic construct
aaggctctgggccgcaaaataaac

SEQ ID NO: 6 - DNA, 23 bp, Artificial Sequence
  Synthetic construct
ccaactaaaaaggccccgaaaaa

SEQ ID NO: 7 - DNA, 20 bp, Artificial Sequence
  Synthetic construct
gtacgccctggagtgacatc

SEQ ID NO: 8 - DNA, 19 bp, Artificial Sequence
  Synthetic construct
cctcgtaggagacccaagc

SEQ ID NO: 9 - DNA, 20 bp, Artificial Sequence
  Synthetic construct
ctagctccccagcagagagt

SEQ ID NO: 10 - DNA, 20 bp, Artificial Sequence
  Synthetic construct
gtggtgcagggacttagctc

SEQ ID NO: 11 - DNA, 24 bp, Artificial Sequence
  Synthetic construct
ggctggtcggctgctctgggaaca

SEQ ID NO: 12 - DNA, 25 bp, Artificial Sequence
  Synthetic construct
ggtggtgggggtagctggcgtctgt